(19) United States
(12) Patent Application Publication
(10) Pub. No.: US 2023/0056824 A1
(43) Pub. Date: Feb. 23, 2023

(54) COSMETIC PRODUCT CAKE WITH ECOLOGICAL SUPPORT AND MANUFACTURING PROCESS THEREOF

(71) Applicant: INTERCOS S.p.A., Milano (IT)

(72) Inventors: Ibrahim HANNO, GORGONZOLA (MI) (IT); Donato SMALDONE, PARABIAGO (MI) (IT); Gabriele DEPTA, MONZA (MB) (IT); Sara BETTINELLI, PARABIAGO (MI) (IT)

(73) Assignee: INTERCOS S.p.A., Milano (IT)

(21) Appl. No.: 17/796,019

(22) PCT Filed: Jan. 27, 2021
(86) PCT No.: PCT/EP2021/051811

§ 371 Date: Jul. 28, 2022

(30) Foreign Application Priority Data
Jan. 29, 2020 (IT) 102020000001663

(51) Int. Cl. A61K 8/02; A61K 8/64; A61K 8/73; A61K 8/60; A61K 8/36; A61K 8/26

(52) U.S. Cl. CPC A61K 8/0233; A61K 8/645; A61K 8/731; A61K 8/732; A61K 8/60; A61K 8/36; A61K 8/26; A61K 2800/805; A61K 2800/87

(57) ABSTRACT

The production of a cosmetic cake (8) is described, consisting of a base supporting and anchoring layer (6) and an overlapping cosmetic product layer (4), wherein said base layer (6) is formed using a dried and solidified aqueous mixture of only natural non-synthetic ingredients.

The present invention relates to a cosmetic product cake with ecological support and to the manufacturing process thereof.

Plastic consumption has risen from 15 million tons in 1964 to more than 310 million tons today. Plastic waste now pollutes a large part of natural environments. Some studies estimate that there are up to 150 tons of plastic in the oceans and that if the production trend continues in the current manner, plastic could reach 34 billion tons by 2050, at least 12 tons of which would be waste scattered throughout the environments. These figures suggest how fundamental the recycling or replacement of plastic objects is, with the aim of achieving a radical decrease in the production of this material.

The possibility to replace plastic both as an ingredient and as primary and secondary packaging is a process underway in all industrial and non-industrial areas. In cosmetics, the replacement of raw materials which generate microplastics is already a process which has been underway for some time, with first significant results.

EP 1078625 A2 describes a solid or viscous cosmetic composition enclosed in a plastic sheath containing solid proteins, liquid or solution polyols, solid polyols, and softeners.

It is the object of the present invention to obtain, with a different non-plastic material, the so-called anchoring grids made of plastic material which are usually used in large volumes (about 2 million pieces per month, therefore with high consumption of plastic) for providing cakes of cosmetic products to be anchored inside a container.

In order to achieve the aforesaid object, the present invention suggests a cosmetic cake consisting of a layer of cosmetic product and an underlying base layer for supporting and anchoring said cosmetic product layer inside a container, characterized in that said base layer consists of a dried and solidified aqueous mixture of only natural non-synthetic ingredients.

The aforesaid aqueous mixture preferably includes the following formulation:

TABLE 1

| Ingredients | Percentages by weight |
|---|---|
| water | from 20.00% to 80.00% |
| organic acids | from 0.05% to 2.00% |
| cellulose or derivatives | from 1.00% to 10.00% |
| vegetal proteins | from 0.10% to 20.00% |
| sugars | from 1.00% to 20.00% |
| starches or dextrins | from 10.00% to 70.00% |
| mica | from 10.00% to 20.00% |
| preservatives | from 0.20% to 1.00% |

and even more preferably the formulation indicated in the following table:

TABLE 2

| Ingredients | Percentages by weight |
|---|---|
| water | 59.20% |
| organic acids | 0.20% |
| cellulose or derivatives | 1.00% |
| vegetal proteins | 1.00% |
| sugars | 8.10% |
| starches or dextrins | 15.50% |
| mica | 14.00% |
| preservatives | 1.00% |

The exercise carried out was to transfer the use of natural ingredients already used for some time in the food industry as alternative packaging to polystyrene or plastic from food technology to cosmetics.

The formula starts from a food wafer recipe, in which the flour has been replaced by natural mica and the protein part thereof (gluten) has been replaced by vegetal proteins (pea, lentil, bean, broad bean, etc.), now easily available in the market of ingredients for cosmetic products.

The bearing structure is ensured by, in addition to proteins, cellulose or hydrocolloids derived from cellulose (crystalline cellulose, methylcellulose, hydroxypropyl cellulose, hydroxypropyl methylcellulose, carboxymethyl cellulose). These hydrocolloids are activated in water under strong stirring until a foam is obtained.

The sugars present in the formula are the third key ingredient which allows stabilizing the foam created during the preparation of the mixture and after drying. They make the structure very rigid and capable of being perfectly anchored to the cosmetic product to be supported. These sugars can be monosaccharides, disaccharides, or polysaccharides of vegetable origin (maize, rice, tapioca, wheat, or legumes). The origin thereof can be starch which is dextrinized enzymatically.

Starch as it is or modified is used as a filling agent in combination with natural mica. Mica gives the substrate a white color and controls the absorption of water.

Citric acid, sorbic acid, benzoic acid, and hydrochloric acid are preferably used as organic acids.

Preservatives, for example caprylyl glycol, hexylene glycol, ethylhexyl glicerine, ethylparaben, methylparaben, propylparaben, phenoxyethanol, sodium benzoate, potassium sorbate and/or sodium dehydroxyacetate, are used since the starting formula contains a high amount of water, which is to be preserved.

It has thus been seen that it is possible to replace the usual anchoring grids made of plastic material by corresponding support and anchoring elements made of a natural material, also called “natural grids”, by pouring an aqueous fluid mixture with ingredients only of natural, non-synthetic origin as described above on a layer of cosmetic product (powder or fluid) previously introduced into a mold and inserting the whole in an oven at 60° C., where the aqueous mixture dries and becomes rigid, irreversibly adhering to the cosmetic product layer, which is in turn solidified.

It was noted that:
1. The support base or natural grid firmly attaches to all types of cosmetic products.
2. Whether the product has been gelled for a few minutes or after 1 day, therefore cold by now, there are no “sticking” defects.
3. The mixture of the natural grid is cold processed, not containing raw materials which are to be activated with temperature; therefore, no energy costs are added to the process.
4. The natural grid mixture is fluid and therefore spreads easily over the surface of the cosmetic product layer.
5. The structure of the natural grid is obtained by drying it in a stove or oven at temperatures above 50° C.
6. From a production point of view, it is no longer necessary to use an operator to insert the plastic grid, as the mixture of the natural grid, being fluid, is dosed directly by a volumetric dosing device.
7. It occurs that the plastic grid traces the shape thereof on the main face of the product. This defect disappears with the natural grid.
8. After drying, the complete cake is extracted simply by overturning the mold.
9. The production costs of the molds are eliminated when new formats must be created.
10. The dry substrate is compatible with hot glues.
11. At the production level, the preparation of the mixture with which the natural grid is obtained can be carried out in a single workstation, in large amounts, and then distributed on the individual lines. Each machine needs to be provided with a simple volumetric dosing device.
12. The cosmetic product with a natural support base passes the tests which are performed before the product is marketed (tilting and dripping).

The application features of the natural grid are comparable to those of plastics, with some additional advantages:
it gives a geometric shape to the cosmetic product;
it allows manipulation during the packaging;
it allows gluing the cosmetic product to the primary packaging, as the plastic grid does;
the cosmetic product remains firmly glued to the packaging, allowing it to pass quality control tests;
the cosmetic product does not undergo changes in the shape or structure thereof over time;
the support base or natural grid is an inert material, which does not transfer any of the ingredients thereof to the cosmetic product;
it allows a “plastic free” declaration when the primary packaging becomes a cardboard or biodegradable case.

For better understanding, an example of a process for creating a cake according to the present invention will be described below.

For the description of the process, reference will be made to the accompanying drawings, in which:

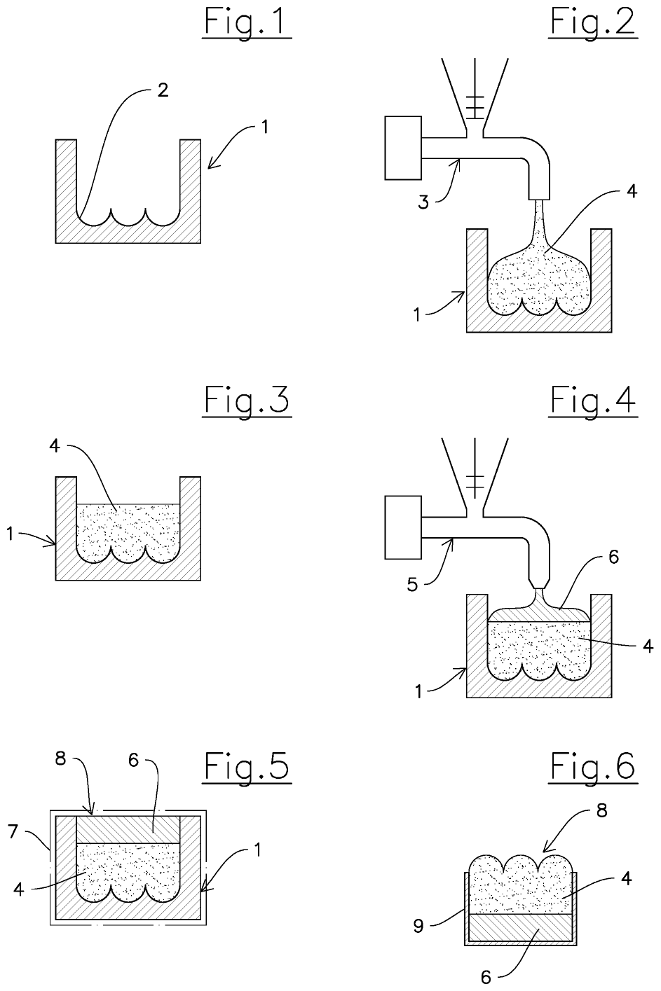

FIG. 1 shows the mold before filling;

FIG. 2 shows the step of filling the mold with a cosmetic product;

FIG. 3 shows the mold filled with a layer of cosmetic product;

FIG. 4 shows the step of pouring the previously prepared aqueous fluid mixture onto the layer of cosmetic product;

FIG. 5 shows the mold and the contents thereof inside a drying and solidifying oven;

FIG. 6 shows the solidified set of the cake formed by the cosmetic product and the support base or grid of natural ingredients, after it has been overturned and placed inside a container.

The process includes, as a preparatory step, the creation of an aqueous fluid mixture consisting only of ingredients of natural, non-synthetic origin, preferably those indicated in the previous table 1, even more preferably with the percentages indicated in table 2.

An appropriate mold is then selected, such as that indicated by reference numeral 1 as a whole in FIG. 1, which has the inner bottom wall 2 shaped complementarily to the desired aesthetic shape for the upper surface of the cosmetic product cake which is to be obtained.

With a suitable dosing device 3 (FIG. 2), a dosed amount of cosmetic product 4, in powder or fluid form, is then introduced into the mold 1, possibly subjected to a slight pressure from above to form a layer of uniform height such as that shown in FIG. 3.

A further dosing device 5 (FIG. 4) then pours a suitable dose of the previously prepared aqueous fluid mixture 6 on the cosmetic product layer 4, where by virtue of the fluid state thereof, it spreads so as to form a substantially uniform fluid mixture layer 6.

The mold 1 with the cosmetic product layer 4 and the overlapping fluid mixture layer 6 is then placed in an oven at 60° C., where the drying of the aqueous part of the fluid layer 6 occurs along with the formation of a solidified cosmetic cake 8 consisting of a lower layer 4 of solidified cosmetic product and an upper layer 6 consisting of a solidified mixture of natural ingredients.

The cake 8 is finally overturned and introduced into a container 9 where the cake itself consists of a base support and anchoring layer 6 (also referred to as a “grid”) of a natural type, without plastic ingredients, and of an overlapping layer 4 of solidified cosmetic product with a shaped upper surface, firmly anchored to said support and anchoring base 6.

1-14. (canceled)

15. A cosmetic cake intended for insertion into a container having a bottom base and lateral walls, wherein the cosmetic cake consists of an upper layer of cosmetic product and an underlying lower layer attached below said upper layer for supporting said upper layer and anchoring said upper layer to the bottom base of the container, wherein said supporting and anchoring lower layer consists of a dried and solidified aqueous mixture consisting of water and solid ingredients, wherein all said solid ingredients are non-synthetic ingredients.

16. A cosmetic cake according to claim 15, wherein said aqueous mixture has the following formulation:
a) water from 20.00% to 80.00% by weight;
b) organic acids from 0.05% to 2.00% by weight;
c) cellulose or derivatives from 1.00% to 10.00% by weight;
d) vegetal proteins from 0.10% to 20.00% by weight;
e) sugars from 1.00% to 20.00% by weight;
f) starches or dextrins from 10.00% to 70.00% by weight;
g) mica from 10.00% to 20.00% by weight; and
h) preservatives from 0.20% to 1.00% by weight.

17. A cosmetic cake according to claim 16, wherein said aqueous mixture has the following formulation:
a) water in an amount of 59.20% by weight;
b) organic acids in an amount of 0.20% by weight;
c) cellulose or derivatives in an amount of 1.00% by weight;
d) vegetal proteins in an amount of 1.00% by weight;
e) sugars in an amount of 8.10% by weight;
f) starches or dextrins in an amount of 15.50% by weight;
g) mica in an amount of 14.00% by weight; and
h) preservatives in an amount of 1.00% by weight.

18. A cosmetic cake according to claim 16, wherein said vegetal proteins are proteins of peas, lentils, beans or broad beans.

19. A cosmetic cake according to claim 16, wherein said mixture comprises water-activated foam hydrocolloids derived from cellulose, in particular crystalline cellulose, methylcellulose, hydroxypropyl cellulose, hydroxypropyl methylcellulose, carboxymethyl cellulose.

20. A cosmetic cake according to claim 16, wherein said sugars are monosaccharide, disaccharide, or polysaccharide sugars of vegetal origin (maize, rice, tapioca, wheat, or legumes).

21. A cosmetic cake according to claim 20, wherein said sugars are derived from enzymatically dextrinized starch.

22. A process for forming a powder cosmetic product cake according to claim 15, wherein the process consists of a succession of the following steps;
a) preparing an aqueous mixture made of water and non-synthetic solid ingredients only,
b) introducing a dosed amount of cosmetic product into a mold for forming a cosmetic product layer on a bottom base of said mold,
c) pouring a dosed amount of said aqueous mixture on said cosmetic product layer in the mold to form an aqueous mixture layer overlying said cosmetic product layer,
d) inserting said mold with said cosmetic product and aqueous mixture layers into a drying and solidifying oven for forming a dried and solidified cosmetic cake consisting of a solid non-synthetic layer and a cosmetic product layer attached on said non-synthetic layer,
e) extracting the mold from said oven,
f) overturning said mold, and
g) extracting the dried and solidified cosmetic cake from said mold.

23. A process according to claim 22, wherein said aqueous mixture has the following formulation:
a) water from 20.00% to 80.00% by weight;
b) organic acids from 0.05% to 2.00% by weight;
c) cellulose or derivatives from 1.00% to 10.00% by weight;
d) vegetal proteins from 0.10% to 20.00% by weight;
e) sugars from 1.00% to 20.00% by weight;
f) starches or dextrins from 10.00% to 70.00% by weight;
g) mica from 10.00% to 20.00% by weight; and
h) preservatives from 0.20% to 1.00% by weight.

24. A process according to claim 23, wherein said aqueous mixture has the following formulation:
a) water in an amount of 59.20% by weight;
b) organic acids in an amount of 0.20% by weight;
c) cellulose or derivatives in an amount of 1.00% by weight;
d) vegetal proteins in an amount of 1.00% by weight;
e) sugars in an amount of 8.10% by weight;
f) starches or dextrins in an amount of 15.50% by weight;
g) mica in an amount of 14.00% by weight; and
h) preservatives in an amount of 1.00% by weight.
